(19) United States
(12) Patent Application Publication
(10) Pub. No.: US 2015/0291964 A1
(43) Pub. Date: Oct. 15, 2015

(54) Methods and Compositions for treatment of cancer by inhibition of NR2F6

(71) Applicant: Christine Victoria Ichim, Spring Valley, CA (US)

(72) Inventor: Christine Victoria Ichim, Spring Valley, CA (US)

(21) Appl. No.: 14/571,262

(22) Filed: Dec. 15, 2014

Related U.S. Application Data
(63) Continuation-in-part of application No. 13/652,395, filed on Oct. 15, 2012, now Pat. No. 9,091,696.
Application No. 13/652,395 is a continuation-in-part of application No. 12/619,290, filed on Nov. 16, 2009, now abandoned.
(60) Provisional application No. 61/114,764, filed on Nov. 14, 2008.

(51) Int. Cl. C12N 15/113; A61K 45/06; A61K 31/7088

(52) U.S. Cl. CPC C12N 15/1138; A61K 31/7088; A61K 45/06; C12N 2310/14; C12N 2310/11

(57) ABSTRACT

The current invention discloses compositions of matter, protocols and methods of use of treatment for cancer and other diseases of aberrant cellular proliferation and differentiation by inhibiting expression of NR2F6 or activity thereof. In one embodiment, administration of synthetic oligonucleotides that induce RNA interference mediated degradation of the nuclear receptor NR2F6 in human or animal patients is performed at a sufficient concentration or frequency to achieve regression of tumor.

### CROSS-REFERENCE TO RELATED APPLICATIONS

This application claims priority to and is a continuation-in-part to pending Non-Provisional U.S. application Ser. No. 13/652,395 filed Oct. 15, 2012, which claims priority to Non-Provisional U.S. application Ser. No. 12/619,290, filed Nov. 16, 2009, which claims the benefit under 35 USC §119(e) of U.S. provisional application No. 61/114764 filed Nov. 14, 2008, each of which is hereby expressly incorporated by reference in their entirety.

The instant application contains a Sequence Listing which has been submitted in ASCII format via EFS-Web and is hereby incorporated by reference in its entirety. Said ASCII copy, created on Apr. 6, 2015, is named SeqListing14571262.txt and is 16 kilobytes in size.

### FIELD OF THE INVENTION

The invention pertains to the field of cancer therapeutics, more particularly the invention pertains to the utilizing of gene silencing technologies, more specifically pertaining to suppression of the nuclear receptor NR2F6 using compositions that induce RNA interference for use as cancer stem cell inhibitors as well as cancer stem cell pathway inhibitors; to methods of using such compounds to treat cancer; to methods of using such compounds to treat disorders in a mammal related to aberrant NR2F6 pathway activity; to pharmaceutical compositions containing such compounds.

### BACKGROUND

The cancer stem cell model proposes that each cancer consists of a small population of cells capable of unlimited growth and self-renewal, known as cancer stem cells, and a much larger population of cells, descendants of the cancer stem cells, that have lost self-renewal capacity and are undergoing terminal differentiation [1]. Evidence supporting this model has been reported for several malignancies including acute myeloid leukemia [2], brain cancer [3, 4] and breast cancer [5]. The cancer stem cell model has important implications for cancer therapy; eradication of cancer stem cells, the cells responsible for maintenance of the neoplasm, would be necessary and sufficient to achieve cure. Moreover, targeting therapy at the disease stem cell promises a high degree of specificity and, by extension, fewer adverse effects. Anti-cancer stem cell therapy is, of course, predicated on the identification of druggable cancer stem cell-specific targets.

Despite the importance of self-renewal in hematopoietic stem cells (HSC) and cancer biology, the mechanisms governing this function are poorly understood. Progress in this area has been hindered by the scarcity of HSCs within haematopoietic tissue, and by challenges faced in purifying HSCs to the extent necessary for studies of transcription or proteomics. Nonetheless, roles in self-renewal have been identified for several proteins. These include pathways involved in embryonic development (Wnt/b-catenin [6], Notch/Delta-like [7], BMP/SMADs [8]), the hox genes and their partners (Cdx [9], Hoxa9 [10], Hoxa10 [11], Hoxb4 [12], Meis [9], Pbx [9]), and polycomb/trithorax group genes (Bmi1 [13, 14], Mll [15]). In addition, a number of transcription factors involved in blood cell differentiation have also been shown to be necessary for self-renewal (Gata-2 [16], Gfi1 [17], JunB [18], Pu.1 [19], Myb [20], Cbp [21], Myc [22], and Zfx [23]). How these diverse pathways are integrated in vivo is not understood; it has been postulated that epigenetic modifications such as chromatin and histone methylation and acetylation play a key role [24], and that the switch between HSC self-renewal and differentiation is regulated by competition between transcription factor complexes, akin to the interplay among Gata-1, c/ebpa, and Pu.1 that mediates the myeloid/erythroid lineage decision [25, 26].

While progress has been made in studying the self-renewal program initiated by normal haematopoietic stem cells, progress remains limited with respect to human leukemia and cancer stem cells, owing in large part to the difficulty of prospectively isolating human cancer stem cells to homogeneity. Development of targeted therapies treating cancer by eradicating the cancer stem cell is hence limited by the ability to identify drug targets specific to the cancer stem cell. Numerous attempts have been made to isolate pure populations of clonogenic cells by fluorescence activated cell sorting based on cellular immunophenotype. While these experiments successfully enrich for human leukaemia cells with clonal longevity, they fail to isolate pure clonogenic cells[2, 27, 28], i.e. even in the “purified” population clonogenic cells are far outnumbered by contaminating non-clonogenic cells, precluding genetic analysis. Therefore characterization of the transcriptome of clonogenic cancer cells has awaited the development of techniques and approaches that permit the study of homogenous populations of clonogenic versus non-clonogenic cells.

Efforts have focused on finding specific markers that distinguish cancer stem cells from the bulk of the tumor. Markers originally associated with normal adult stem cells have been found to also mark cancer stem cells and co-segregate with the enhanced tumorigenicity of cancer stem cells. The most commonly expressed surface markers by the cancer stem cells include CD44, CD133, and CD166 [27-33]. Sorting tumor cells based primarily upon the differential expression of these surface marker(s) have accounted for the majority of the highly tumorigenic cancer stem cells described to date. Therefore, these surface markers are well validated for identification and isolation of cancer stem cells from the cancer cell lines and from the bulk of tumor tissues, but they do not yield a pure population of cancer stem cells for analysis, because of the possibility of contamination with normal tissues stem cells.

Since surviving cancer stem cells can repopulate the tumor and cause relapse, it would be possible to treat patients with aggressive, non-resectable tumors and refractory or recurrent cancers, as well as prevent the tumor metastasis and recurrence by selectively targeting cancer stem cells. The clinical benefits of developing inhibitors of cancer stem cells holds great hope for improvement of survival and quality of life of cancer patients, especially for sufferers of metastatic disease. The key to unlocking this untapped potential is the identification and validation of pathways that are selectively important for cancer stem cell self-renewal and survival and devising means to inhibit these. Though multiple pathways underlying tumorigenesis in cancer and in embryonic stem cells or adult stem cells have been elucidated in the past, at present, in the art, therapeutics targeting the cancer stem cell is difficult.

While treatment options for some cancers has improved in the last few decades, therapy for other cancers, such as acute myeloid leukemia has not changed significantly in 40 years, and is far from optimal. In acute myeloid leukemia complete remission is achieved in 50-70% of patients, but post-remission therapy, comprising further cycles of intensive chemotherapy or stem cell transplantation, is essential to prevent disease relapse. In the majority of cases chemoresistant clones eventually emerge; overall, cure is achieved in fewer than 30% of patients[29]. Outcomes in patients over 60 years old, who comprise more than half of all cases of acute myeloid leukemia, are even poorer, with cure rates of no more than 5%[29]. In order to improve efficacy and reduce toxicity of acute myeloid leukemia treatment, new therapies must be devised that target the specific cells responsible for the maintenance and expansion of the leukaemic clone—the leukaemia stem cell.

### SUMMARY OF THE INVENTION

The invention provides means of treating cancer through inhibition of the expression and/or activity of the NR2F6 gene and/or protein respectively. In one aspect, treatment of cancer is performed by administration of an agent or plurality of agents capable of inhibiting expression of the NR2F6 gene. Said means of inhibition include administration of hammerhead ribozymes, gene editing means such as TALON or CRISPER mediated DNA cleavage, or means capable of inducing RNA interference such as short interfering RNA (siRNA) or induction of DNA directed RNA interference such as short hairpin RNA (shRNA) expressed from a plasmid, viral, or lentiviral vector. Additionally, inhibition of gene activity may be obtained by administration of antisense oligonucleotides.

The invention discloses compositions comprising synthetic oligonucleotide molecules that induce RNA interference of the NR2F6 gene, and methods of treating cancer by blocking expression of the gene NR2F6 using synthetic oligonucleotides that induce RNA interference.

The RNA interference inducing oligonucleotide is one of the following: short interfering nucleic acid (siNA), short interfering RNA (siRNA), double-stranded RNA (dsRNA), micro-RNA (miRNA), and short hairpin RNA (shRNA) molecules.

### BRIEF DESCRIPTION OF THE DRAWINGS

It will be appreciated that the drawings are not necessarily to scale, with emphasis instead being placed on illustrating the various aspects and features of embodiments of the invention, in which:

FIG. 1 shows that NR2F6 is highly expressed in both long and short term haematopoietic stem cells and that expression of NR2F6 in bone marrow from patients with acute myelogenous leukemia (AML), chronic myelomonocytic leukemia (CMML) and myelodysplastic syndrome (MDS) is greater compared to control. * denotes p<0.05 and ** denotes p<0.01 relative to normal (ANOVA & Tukey post-hoc test).

FIG. 2 shows that expression of NR2F6 is greater in all types of ovarian cancer as determined by in silico analysis.

FIG. 3 shows that expression of NR2F6 is greater in endometrial cancer as determined by in silico analysis.

FIG. 4 shows quantification of NR2F6 (EAR-2) protein levels, determined by immunoblot and quantified using densitometry, in human U937 leukemia cells that were treated with NR2F6 shRNA or a hairpin control.

FIG. 5 shows cytospins that demonstrate morphologically that knock down of NR2F6 using short-hairpin RNAs induces terminal differentiation and blood cell maturation of U937 human leukemia cells.

FIG. 6 shows cytospins from a second experiment that demonstrate morphologically that knock down of NR2F6 using short-hairpin RNAs induces terminal differentiation and blood cell maturation of U937 human leukemia cells.

FIG. 7 shows dot plots generated by flow cytometry showing that knock down of NR2F6 using short-hairpin RNAs induces terminal differentiation and blood cell maturation of U937 human leukemia cells. These data demonstrate that knockdown of NR2F6 was sufficient to allow the leukemia cells to become mature granulocytes blood cells.

FIG. 8 shows histograms of annexin V staining generated by flow cytometry showing that knock down of NR2F6 using short-hairpin RNAs induces terminal differentiation and blood cell maturation of U937 human leukemia cells that is followed spontaneously by apoptosis (programmed cell death).

### DETAILED DESCRIPTION OF THE ILLUSTRATED EMBODIMENTS

Embodiments of the present invention are described below. It is, however, expressly noted that the present invention is not limited to these embodiments, but rather the intention is that modifications that are apparent to the person skilled in the art and equivalents thereof are also included.

The term “NR2F6” as used herein refers to nuclear receptor subfamily2, group F, member 6 and is also referred to as v-erbA-related gene or ear-2 and includes, without limitation, the protein encoded by the gene having the sequence as shown in SEQ ID NO: 1 (human) or SEQ ID NO: 2 (mouse) or variants thereof and the protein having the amino acid sequence as shown in SEQ ID NO: 3 (human) or SEQ ID NO: 4 (mouse) or variants thereof.

The term “a cell” as used herein includes a plurality of cells and refers to all types of cells including hematopoietic and cancer cells. Administering a compound to a cell includes in vivo, ex vivo and in vitro treatment.

The term “stem cell” as used herein refers to a cell that has the ability for self-renewal. Non-cancerous stem cells have the ability to differentiate where they can give rise to specialized cells.

The term “effective amount” as used herein means a quantity sufficient to, when administered to an animal, effect beneficial or desired results, including clinical results, and as such, an “effective amount” depends upon the context in which it is being applied. For example, in the context of inhibiting self-renewal of stem cells, it is the amount of the NR2F6 inhibitor sufficient to achieve such an inhibition as compared to the response obtained without administration of the NR2F6 inhibitor.

The term “oligonucleotide” is intended to include unmodified DNA or RNA or modified DNA or RNA. For example, the nucleic acid molecules or polynucleotides of the disclosure can be composed of single- and double stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning.

The term “animal” as used herein includes all members of the animal kingdom, preferably mammal. The term “mammal” as used herein is meant to encompass, without limitation, humans, domestic animals such as dogs, cats, horses, cattle, swine, sheep, goats, and the like, as well as wild animals. In an embodiment, the mammal is human.

The term “interfering RNA” or “RNAi” or “interfering RNA sequence” refers to double-stranded RNA (i.e., duplex RNA) that targets (i.e., silences, reduces, or inhibits) expression of a target gene (i.e., by mediating the degradation of mRNAs which are complementary to the sequence of the interfering RNA) when the interfering RNA is in the same cell as the target gene. Interfering RNA thus refers to the double stranded RNA formed by two complementary strands or by a single, self-complementary strand. Interfering RNA typically has substantial or complete identity to the target gene. The sequence of the interfering RNA can correspond to the full length target gene, or a subsequence thereof. Interfering RNA includes small-interfering RNA″ or “siRNA,” i.e., interfering RNA of about 15-60, 15-50, 15-50, or 15-40 (duplex) nucleotides in length, more typically about, 15-30, 15-25 or 19-25 (duplex) nucleotides in length, and is preferably about 20-24 or about 21-22 or 21-23 (duplex) nucleotides in length (e.g., each complementary sequence of the double stranded siRNA is 15-60, 15-50, 15-50, 15-40, 15-30, 15-25 or 19-25 nucleotides in length, preferably about 20-24 or about 21-22 or 21-23 nucleotides in length, and the double stranded siRNA is about 15-60, 15-50, 15-50, 15-40, 15-30, 15-25 or 19-25 preferably about 20-24 or about 21-22 or 21-23 base pairs in length). siRNA duplexes may comprise 3′ overhangs of about 1 to about 4 nucleotides, preferably of about 2 to about 3 nucleotides and 5′ phosphate termini. The siRNA can be chemically synthesized or maybe encoded by a plasmid (e.g., transcribed as sequences that automatically fold into duplexes with hairpin loops). siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., PNAS USA 99: 9942-7 (2002); Calegari et al., PNAS USA 99: 14236 (2002); Byrom et al., Ambion TechNotes 10(1): 4-6 (2003); Kawasaki et al., Nucleic Acids Res. 31: 981-7 (2003); Knight and Bass, Science 293: 2269-71 (2001); and Robertson et al., J. Biol. Chem.243: 82 (1968)). Preferably, dsRNA are at least 50 nucleotides to about 100, 200, 300, 400 or 500 nucleotides in length. A dsRNA may be as long as 1000, 1500, 2000, 5000 nucleotides in length, or longer. The dsRNA can encode for an entire gene transcript or a partial gene transcript.

The term “siRNA” refers to a short inhibitory RNA that can be used to silence gene expression of a specific gene. The siRNA can be a short RNA hairpin (e.g. shRNA) that activates a cellular degradation pathway directed at mRNAs corresponding to the siRNA. Methods of designing specific siRNA molecules or shRNA molecules and administering them are known to a person skilled in the art. It is known in the art that efficient silencing is obtained with siRNA duplex complexes paired to have a two nucleotide 3′ overhang. Adding two thymidine nucleotides is thought to add nuclease resistance. A person skilled in the art will recognize that other nucleotides can also be added.

The term “antisense nucleic acid” as used herein means a nucleotide sequence that is complementary to its target e.g. a NR2F6 transcription product. The nucleic acid can comprise DNA, RNA or a chemical analog, that binds to the messenger RNA produced by the target gene. Binding of the antisense nucleic acid prevents translation and thereby inhibits or reduces target protein expression. Antisense nucleic acid molecules may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

As used in this context, to “treat” means to ameliorate at least one symptom of the disorder. In some embodiments, a treatment can result in a reduction in tumor size or number, or a reduction in tumor growth or growth rate.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and origin.

As used herein, the terms “cancer”, “hyperproliferative” and “neoplastic” refer to cells having the capacity for autonomous growth, i,e., an abnormal state or condition characterized by rapidly proliferating cell growth. Hyperproliferative and neoptastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, e.g., affecting the nervous system, lung, breast, thyroid, lymphoid, gastrointestinal, and genito-urinary tract, as well as adenocarcinomas, which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

The invention provides methods for treating a cellular proliferative disorder, such as neoplasia, in a mammalian subject (eg. rodent such as mouse, or primate such as human, chimpanzee or monkey). The methods include selecting a subject who is in need of treatment for a cellular proliferative disorder or a disorder of cellular differentiation, administering to the subject a therapeutically effective amount of an oligonucleotide that activates the RNA inference pathway against the gene target NR2F6, thereby treating the cellular proliferative disorder or the disorder of cellular differentiation in the subject. Disorders of cellular proliferation and differentiation is selected from the group consisting of neoplasia (cancer), hyperplasias, restenosis, cardiac hypertrophy, immune disorders and inflammation. Preferably, said cell proliferative disorder is a neoplastic disorder, i.e., cancer. In some embodiments, the cancer includes, but is not limited to papilloma, blastoglioma, Kaposi's sarcoma, melanoma, lung cancer, ovarian cancer, prostate cancer, squamous cell carcinoma, astrocytoma, head cancer, neck cancer, bladder cancer, breast cancer, lung cancer, colorectal cancer, thyroid cancer, pancreatic cancer, gastric cancer, hepatocellular carcinoma, leukemia, lymphoma, Hodgkin's disease, osteosarcoma, testicular cancer, and Burkitt's disease. In one embodiment of the invention the oligonuclotides are used to induce a reduction of proliferation of the cancer cells. In another embodiment of the invention the oligonucleotides are used to induce the differentiation of the cancer cells. In yet another embodiment of the invention the oligonucleotides are used to specifically target the functions of the cancer stem cells.

One embodiment of the invention is a short-interfering ribonucleic acid (siRNA) molecule effective at silencing NR2F6 expression or substantially inhibiting NR2F6 expression. In one embodiment of the invention the oligonucleotide backbone is chemically modified to increase the deliverability of the interfering ribonucleic acid molecule. In another embodiment these chemical modifications act to neutralize the negative charge of the interfering ribonucleic acid molecule. One embodiment of the invention consists of a pharmaceutical composition comprising an siRNA oligonucleotide that induces RNA interference against NR2F6. It is known to one of skill in the art that siRNAs induce a sequence-specific reduction in expression of a gene by the process of RNAi, as previously mentioned. Thus, siRNA is the intermediate effector molecule of the RNAi process that is normally induced by double stranded viral infections, with the longer double stranded RNA being cleaved by naturally occurring enzymes such as DICER. Some nucleic acid molecules or constructs provided herein include double stranded RNA molecules comprising 16-30, e.g., 16, 17, 18, 19, 20, 21, 22, 23, 24, 25, 26, 27, 28, 29, or 30 nucleotides in each strand, wherein one of the strands is substantially identical, for example at least 85% (or more, as for example, 90%, 95%, or 100%) identical, e.g., having 3, 2, 1, or 0 mismatched nucleotide(s), to a target region in the mRNA of NR2F6 and the other strand is identical or substantially identical to the first strand. However, it will be appreciated that the dsRNA molecules may have any number of nucleotides in each strand which allows them to reduce the level of NR2F6 protein, or the level of a nucleic acid encoding NR2F6 . The dsRNA molecules provided herein can be chemically synthesized, or can be transcribed in vitro from a DNA template, or in vivo from, e.g., shRNA, which is mentioned below. The dsRNA molecules can be designed using any method known in the art.

In one embodiment, nucleic acids provided herein can include both unmodified siRNAs and modified siRNAs as known in the art. For example, in some embodiments, siRNA derivatives can include siRNA having two complementary strands of nucleic acid, such that the two strands are crosslinked. For a specific example, a 3′ OH terminus of one of the strands can be modified, or the two strands can be crosslinked and modified at the 3′ OH terminus. The siRNA derivative can contain a single crosslink (one example of a useful crosslink is a psoralen crosslink). In some embodiments, the siRNA derivative has at its 3′ terminus a biotin molecule (for example, a photocleavable molecule such as biotin), a peptide (as an example an HIV Tat peptide), a nanoparticle, a peptidomimetic, organic compounds, or dendrimer. Modifying siRNA derivatives in this way can improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acids described within the practice of the current invention can include nucleic acids that are unconjugated or can be conjugated to another moiety, such as a nanoparticle, to enhance a desired property of the pharmaceutical composition. Properties useful in the development of a therapeutic agent include: a) absorption; b) efficacy; c) bioavailability; and d) half life in blood or in vivo. RNAi is believed to progress via at least one single stranded RNA intermediate, the skilled artisan will appreciate that single stranded-siRNAs (e.g., the antisense strand of a ds-siRNA) can also be designed as described herein and utilized according to the claimed methodologies.

In one embodiment the pharmaceutical composition comprises a nucleic acid-lipid particle that contains an siRNA oligonucleotide that induces RNA interference against NR2F6. In some aspects the lipid portion of the particle comprises a cationic lipid and a non-cationic lipid. In some aspects the nucleic acid-lipid particle further comprises a conjugated lipid that prevents aggregation of the particles and/or a sterol (e.g., cholesterol).

For practice of the invention, methods for expressing siRNA duplexes within cells from recombinant DNA constructs to allow longer-term target gene suppression in cells are known in the art, including mammalian Pol III promoter systems (e.g., H1 or U6/snRNA promoter systems) capable of expressing functional double-stranded siRNAs. Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by an H1 or U6 snRNA promoter can be expressed in cells, and can inhibit target gene expression. Constructs containing siRNA sequence(s) under the control of a T7 promoter also make functional siRNAs when co-transfected into the cells with a vector expressing T7 RNA polymerase. A single construct may contain multiple sequences coding for siRNAs, such as multiple regions of the NR2F6 gene, such as a nucleic acid encoding the NR2F6 mRNA, and can be driven, for example, by separate Pol III promoter sites. In some situations it will be preferable to induce expression of the hairpin siRNA or shRNAs in a tissue specific manner in order to activate the shRNA transcription that would subsequently silence NR2F6 expression. Tissue specificity may be obtained by the use of regulatory sequences of DNA that are activated only in the desired tissue. Regulatory sequences include promoters, enhancers and other expression control elements such as polyadenylation signals. Regulatory sequences include those which direct constitutive expression of a nucleotide sequence in many types of host cell and those which direct expression of the nucleotide sequence only in certain host cells. Tissue specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. Similarly, promoters as follows may be used to target gene expression in other tissues. Examples of more tissue specific promoters include in (a) to target the pancreas promoters for the following may be used: insulin, elastin, amylase, pdr-I, pdx-I, glucokinase; (b) to target the liver promoters for the following may be used: albumin PEPCK, HBV enhancer, a fetoprotein, apolipoprotein C, .alpha.-I antitrypsin, vitellogenin, NF-AB, Transthyretin; (c) to target the skeletal muscle promoters for the following may be used: myosin H chain, muscle creatine kinase, dystrophin, calpain p94, skeletal .alpha.-actin, fast troponin 1; (d) to target the skin promoters for the following may be used: keratin K6, keratin KI; (e) lung: CFTR, human cytokeratin IS (K 18), pulmonary surfactant proteins A, B and C, CC-10, Pi; (0 smooth muscle: sm22 .alpha., SM-.alpha.-actin; (g) to target the endothelium promoters for the following may be used: endothelin-I, E-selectin, von Willebrand factor, TIE, KDR/flk-I; (h) to target melanocytes the tyrosinase promoter may be used; (i) to target the mammary gland promoters for the following may be used: MMTV, and whey acidic protein (WAP).

Yet another embodiment of the invention consists of a pharmaceutical composition comprising an oligonucleotide that induces RNA interference against NR2F6 combined with a delivery agent such as a liposome. For more targeted delivery immunoliposomes, or liposomes containing an agent inducing selective binding to neoplastic cells may be used.

The present invention further provides pharmaceutical compositions comprising the nucleic acid-lipid particles described herein and a pharmaceutically acceptable carrier.

Another embodiment of the invention consists of a pharmaceutical composition comprising an oligonucleotide that induces RNA interference against NR2F6 combined with an additional chemotherapeutic agent.

Yet another embodiment of the invention consists of a pharmaceutical composition comprising an oligonucleotide that induces RNA interference against NR2F6 combined with an additional agent used to induce differentiation

One embodiment of the invention is a short-interfering ribonucleic acid (siRNA) molecule effective at silencing NR2F6 expression that has been cloned in to an appropriate expression vector giving rise to an shRNA vector.

In certain embodiment shRNA olignucleotides are cloned in to an appropriate mammalian expression vectors, examples of appropriate vectors include but are not limited to lentiviral, retroviral or adenoviral vector. In this embodiment, the invention consists of a viral vector, comprising the inhibitory RNA molecule described above. The viral vector preferably is a lentivirus. In one aspect the viral vector is capable of infecting cancer cells. Another embodiment is a lentivirus vector that is an integrating vector. The viral vector preferably is capable of transducing cancer cells. The viral vector is preferably packaged in a coat protein the specifically binds to cancer cells. The viral vector preferably is capable of expressing an RNA that inhibits NR2F6 expression. Another embodiment of the invention is one in which the viral vector is preferably produced by a vector transfer cassette and a separate helper plasmid. In certain embodiment the shRNA olignucleotides is combined with a pharmaceutically acceptable vehicle a pharmaceutical composition. One embodiment is a pharmaceutical composition comprising an inhibitory oligonucleotide that is a double stranded RNA molecule.

One aspect of the invention is a microRNA or family of microRNAs are administered that substantially inhibit expression of NR2F6

In one embodiment, the inhibition of NR2F6 is utilized to enhance efficacy of existing anticancer approaches, or therapies. Specifically, inhibition of NR2F6 may be combined with agents selected from a group comprising of: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anti-cancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bisacridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

The present inventors have found that NR2F6 is a regulator of cancer cell proliferation, self-renewal and differentiation, and that silencing of NR2F6 with oligonucleotides that induce RNA interference induces a reduction of cancer cell proliferation, inhibiting clonogenicity and self-renewal of proliferating cancer cells, and induces differentiation.

Accordingly, the present disclosure provides a method of modulating cancer cell growth, proliferation and/or differentiation comprising administering an effective amount of a synthetic oligonucleotide that induces RNA interference of NR2F6 to a cell or animal in need thereof.

In one aspect, the synthetic oligonucleotide is an siRNA targetting NR2F6. In another aspect, the the synthetic oligonucleotide is an shRNA targeting NR2F6. And yet in another aspect the synthetic oligonucleotide is an antisense RNA molecule targeting NR2F6.

Accordingly, the present disclosure provides a method of inhibiting self-renewal of stem cells comprising administering an effective amount of an oligonucleotides that induce RNA interference to a cell or animal in need thereof. The present disclosure also provides the use of a oligonucleotides that induce RNA interference for inhibiting self-renewal of stem cells in a cell or animal in need thereof. The present disclosure further provides the use of an oligonucleotide that induce RNA interference in the preparation of a medicament for inhibiting self-renewal of stem cells in a cell or animal in need thereof. The present disclosure also provides a oligonucleotides that induce RNA interference for use in inhibiting self-renewal of stem cells in a cell or animal in need thereof.

In another embodiment, the present disclosure provides a method of inducing terminal differentiation of stem cells comprising administering of an effective amount of oligonucleotides that induce RNA interference to NR2F6 to a cell or animal in need thereof. The present disclosure also provides the use of oligonucleotides that induce RNA interference to NR2F6 for inducing terminal differentiation of stem cells in a cell or animal in need thereof. The present disclosure further provides the use of oligonucleotides that induce RNA interference to NR2F6 in the preparation of a medicament for inducing terminal differentiation of stem cells in a cell or animal in need thereof. The present disclosure also provides oligonucleotides that induce RNA interference to NR2F6 for use in inducing terminal differentiation of stem cells in a cell or animal in need thereof.

In one embodiment, the stem cells are cancer stem cells, leukemia stem cells or ovarian cancer stem cells.

The term “inhibiting self renewal of stem cells” as used herein includes but is not limited to preventing or decreasing the clonal longevity, clonogenicity, serial replating ability, clonogenic growth and/or transplantability of the stem cells.

### EXAMPLES

### Materials and Methods

### Cell Lines

U937 and 32Dc13 cells were purchased from ATCC (Manassas, Va.). The 293GPG retroviral packaging cell line was a gift of Richard Mulligan, Harvard University. U937 cells were purchased from ATCC and grown in RPMI supplemented with 10% FBS. 32Dc13 cells were purchased from ATCC and grown in RPMI with 1 ng/mL of rmIL-3. The 293GPG retroviral packaging cell line (a gift of Richard Mulligan, Harvard University) was grown in DMEM medium supplemented with 10% FBS, tetracycline (1 mg/mL), G418 (0.3 mg/mL) and puromycin (2 mg/mL).

Generation of shRNA

Oligonucleotides targeting human or mouse NR2F6 were synthesized (Sigma-Genosys, Oakville, ON Canada), annealed and cloned into the pSiren vector (Clonetech, Mountain View Calif.), after which sequence was verified at The Centre for Applied Genomics (TCAG), Toronto, ON Canada. Virus was prepared by transient transfection of plasmid in the 293GPG cell line as described above.

Sense shRNA hairpin sequences were as follows:

mus shNR2F6.1
5′-GAT CCG CAT TAC GGC GTG TTC ACC TTC AAG AGA GGT
GAA CAC GCC GTA ATG CTT TTT TCT AGA G 3′
mus shNR2F6.2
5′-GAT CCG CAA CCG TGA CTG TCA GAT TAA GTT CTC TAA
TCT GAC AGT CAC GGT TGT TTT TTC TAG AG-3′
mus shNR2F6.3
5′-GAT CCG TGT CCG AGC TGA TTG CGC ATT CAA GAG ATG
CGC AAT CAG CTC GGA CAT TTT TTC TAG AG-3′
human shNR2F6.1
5′-GAT CCG CAT TAC GGT GTC TTC ACC TTC AAG AGA GGT
GAA GAC ACC GTA ATG CTT TTT TCT AGA G-3′
human shNR2F6.2
5′-GAT CCG CCT CTG GAC ACG TAA CCT ATT CAA GAG ATA
GGT TAC GTG TCC AGA GGT TTT TTC TAG AG-3′

Generating shRNA Retrovirus

The 293GPG retroviral packaging cell line (a gift of Richard Mulligan, Harvard University) was grown in DMEM medium supplemented with 10% FBS, tetracycline (1 mg/mL), G418 (0.3mg/mL) and puromycin (2 mg/mL). VSV-G pseudotyped retroviral particles were generated by transient transfection of 293GPG cells. 293GPG cells were cultured in 15cm plates with 30 mL of 293GPG medium. 12 hours after removal of antibiotics, cells were transiently transfected with 25 μg of plasmid DNA using Lipofectamine 2000 (Invitrogen). Virus was collected on days 3 to 7, concentrated by centrifugation at 16,500 RPM for 90 minutes. Transduction of >95% of cells was confirmed by flow cytometry.

Generation of shRNA Lentivirus

The packaging vectors pRSV Rev, pMD2.G (VSV-G) and pMDLg/pRRE, as well as the shRNA vector H1GIP (a kind gift from John Dick, University Health Network) were grown in STBL2 competent cells (Invitrogen, Carlsbad, Calif.) at 30 degrees. Plasmid DNA was extracted using the EndoFree Mega kit (Qiagen).

293T/17 cells were passaged 1:4 to 1:6 three times a week, before reaching 80% confluence. This passaging schedule was intended to maintain the cells at a density where they would be in a log state of proliferation, as well as to maintain them as individual cells (as opposed to cell aggregates) which would also increase transfection efficiency. Only early passages of the 293T/17 cells lines were used for the production of lentivirus, furthermore, batches of cells were not maintained in culture for more than a month. Care was taken to maintain 293T/17 cells endotoxin free.

293T/17 cells were transfected using the CalPhos Mammalian Transfection Kit (Clonetech, Palo Alto, Calif.) in 15 cm plates. Briefly, 12×106 cells were plated in a 15 cm dish the day prior to transfection. Two hours before transfection medium was aspirated and cells were fed 25 mL of fresh medium. Calcium Phosphate precipitates were prepared in 50 mL conical tubes in master mixes sufficient for transfecting 6 plates. Each plate received a solution containing 63.4 μg of DNA (28.26 μg of the H1 shRNA hairpin vector; 18.3 μg of pMDLg/pRRE; 9.86 μg of pMD2.G and 7.04 μg of pRSV Rev) and 229.4 μL of 2 M Calcium solution in a total volume of 3.7 mL. The transfection solution was incubated 20 minutes at room temperature and was then added drop wise to each plate. Plates were incubated overnight with transfection precipitate, and washed with PBS the next morning.

Lentiviral supernatent was collected after 24 and 48 hours. Supernatant was centrifuged in a table-top centrifuge for 10 minutes to remove debris and then pooled and filtered through a 0.45 μm pore size polyethersulfone (PES) bottle-top filter (Nalgene, Thermo Fisher Scientific). Ultracentrifugation was conducted as described above.

### Immunoblotting

Immunoblotting for human NR2F6 was performed using the PP-N2025-00 (Perseus Proteomics, Tokyo, Japan), or ab12982 (Abcam, Cambridge, Mass.) antibodies, while immunoblotting for mouse NR2F6 was performed using the LS-C40527 (LifeSpan Biosciences, Seattle, Wash.) antibody. Western blot analysis. Cells were lysed in RIPA lysis buffer (1% SDS, 1% Triton X-100, 1% deoxycholic acid) and quantified using the DC Protein Assay kit (Bio-Rad). Proteins (25-50 μg) in lysates were resolved on 10% SDS—PAGE gels and transferred to nitrocellulose membrane (Protran, Whatman). The membranes were blocked with 5% non-fat dry milk in 0.1% TBS/Tween-20 or 2% BSA-TBS/Tween-20 (CD95, CD95L and E-cadherin) and incubated in primary antibodies diluted in blocking solution at 4 ° C. overnight. After incubation with secondary antibodies, detection was performed using the ECL method (Amersham Pharmacia Biotech) and developed using a chemiluminescence imager, G:BOX Chemi XT4 (Synoptics).

### Quantitative PCR

RNA was isolated from 1×106 cells using Trizol reagent (Invitrogen, Burlington, ON Canada) and first strand cDNA was synthesized using SuperScript II Reverse Transcriptase (Invitrogen) according to manufacturer's instructions. Real time PCR was performed according to manufacturer's instructions using SYBR Green Master Mix (Applied Biosystems, Foster City, Calif.) and analyzed using the delta-delta CT method. Primer sequences are as follows:

Human NR2F6:
Fwd: 5′-TCTCCCAGCTGTTCTTCATGC-3′
Revs: 5′-CCAGTTGAAGGTACTCCCCG-3′
Human GAPDH:
Fwd: 5′-GGCCTCCAAGGAGTAAGACC-3′
Revs: 5′-AGGGGTCTACATGGCAACTG-3′.
3′ end Mus NR2F6:
Fwd: 5′-CCTGGCAGACCTTCA ACAG-3′
Revs: 5′-GATCCTCCTGGCCCATAGT-3′
3′ end Mus L32:
Fwd: 5′-GCCATCAGAGTCACCAATCC-3′
Revs: 5′-AAACATGCACACAAGCCATC-3′

### Flow Cytometry

For analysis of c-kit+, sca-1+, lineage- (KSL) cells, red blood cell depleted bone marrow cells were stained with a cocktail containing biotin CD3, biotin CD45R/B220 (RA3-6B2), biotin CD11b (M1/70), biotin erythroid marker (TER-119), biotin Ly-6G (RB6-8C5), c-kit APC, sca-1 PE-Cy7 and either CD34 PE or CD49b PE (all eBioscience) in the dark. Bone marrow was washed once and incubated with streptavidin PE-Cy5 for 20 minutes in the dark. Bone marrow was washed twice and analyzed using flow cytometry on a Becton Dickinson LSR II. All samples analyzed were gated based on FSC/SSC and GFP+ cells. The population of KSL cells is highly enriched for hematopoietic stem cell activity. This population was analyzed and further subdivided based on the expression of the CD34 and CD49b antigen.

siRNA Transfection of Cell Lines with siRNA

For siRNA transfection, cells grown in 12-well plates were submitted to lipofection using 6 μl of the HiperFect reagent (Qiagen) and 150 ng/well of either negative control siRNA or NR2F6 siRNA. For each experiment at least four siRNA targeting different sequences were used.

### Cell Death Assays

Different cell death assays were used, depending on specific experimental requirements. To quantify DNA fragmentation after a treatment, both dead and live cells were collected for the assay. The total cell pellet was resuspended in 0.1% sodium citrate, pH 7.4, 0.05% Triton X-100 and 50 μg ml-1 propidium iodide. After 2-4 h in the dark at 4 ° C., fragmented DNA (% subG1 nuclei) was quantified with flow cytometry. To stain cells with DAPI, after a treatment, both dead and live cells were collected and resuspended in 200-300 μl of media, and DAPI was added at 0.025 mg ml-1. Percent dead cells (DAPI-positive) was monitored using FACS in combination with FSC-A and SSC-A gating. To quantify cell death using the trypan blue exclusion assay, cells were resuspended in media and an equal volume of Trypan blue solution (Cellgro) was added. Both living and dead (blue) cells were counted on a haemocytometer under a light microscope. Annexin V staining was performed using apoptosis detection kit from R and D systems.

### Example I

### Augmented Expression of NR2F6 in Neoplastic Tissue

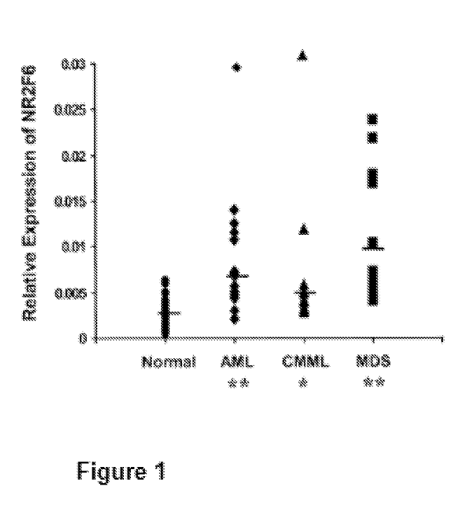

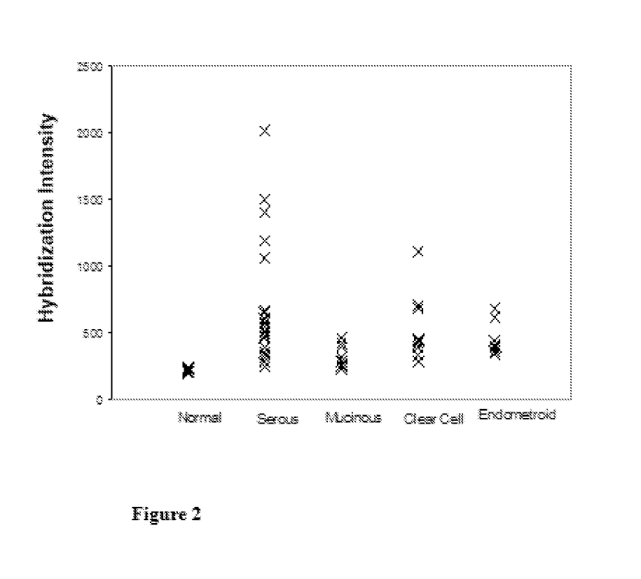

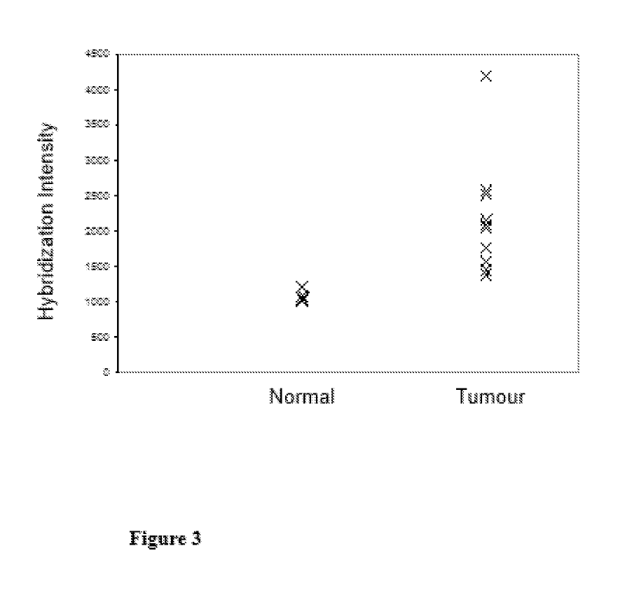

Expression of NR2F6 was consistently upregulated in neoplastic tissues in leukemic (FIG. 1), ovarian cancer (FIG. 2) and endometrial cancer (FIG. 3) as compared to non-malignant tissues.

### Example II

### Knockdown of NR2F6 Induces Differentiation and Apoptosis of U937 Cancer Cells.

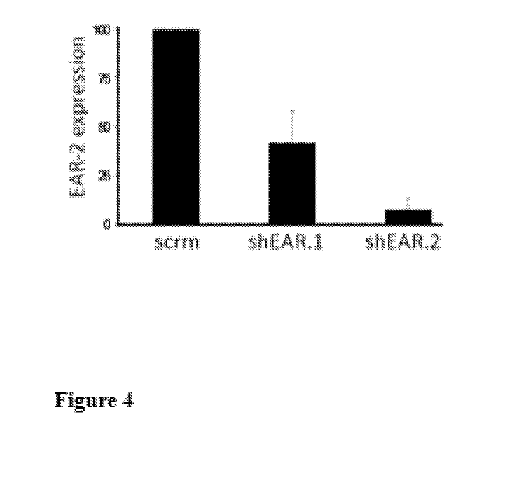

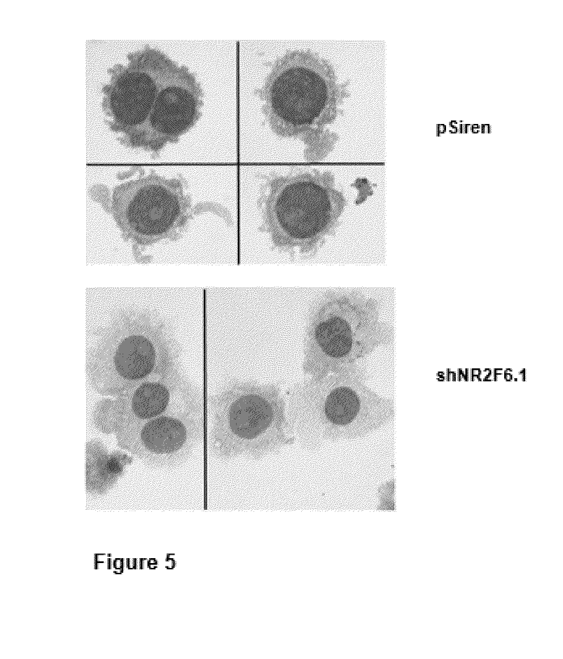

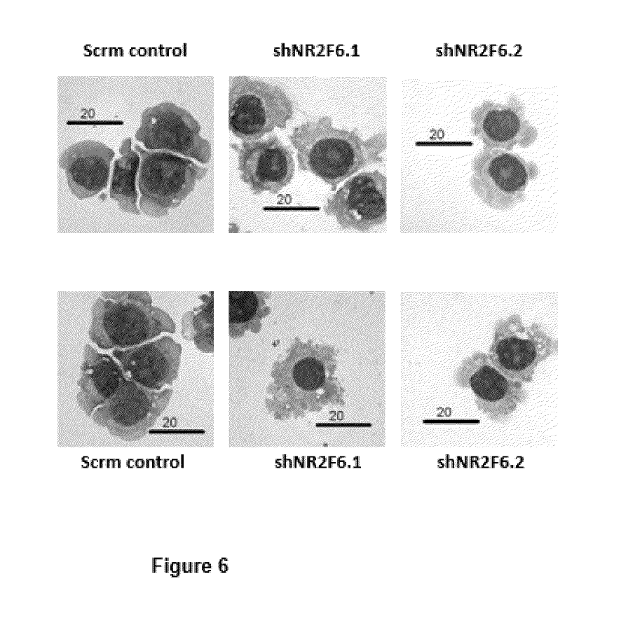

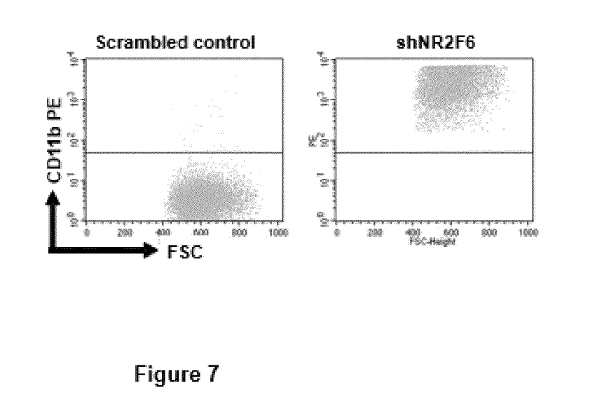

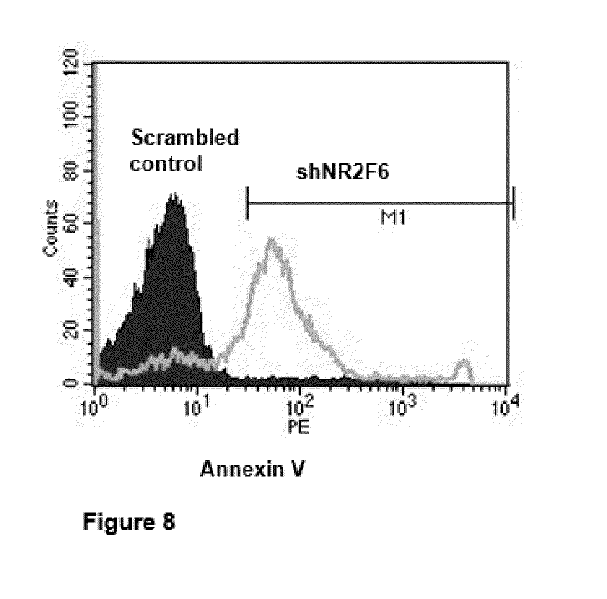

Short hairpin RNA constructs were shown to silence NR2F6 expression in U937 cells (FIG. 4). Knockdown of NR2F6 resulted in differentiation of U937 cells along hematopoietic lineages based on morphology as seen in FIGS. 5 and 6. Flow cytometry examination revealed monocytic differentiation subsequent to NR2F6 silencing based on CD11b staining (FIG. 7). Assessment of apoptosis by Annexin V staining revealed increased apoptosis in cells silenced for NR2F6 (FIG. 8).

### Sequence Listing

SEQ ID NO: 1
NCBI Reference Sequence: NM 005234.3
>gi|46411186|ref|NM_{—}005234.3| Homo sapiens nuclear receptor subfamily 2, group F, member 6 (NR2F6), mRNA

GTGCAGCCCGTGCCCCCCGCGCGCCGGGGCCGAATGCGCGCCGCGTAGGG
TCCCCCGGGCCGAGAGGGGTGCCCGGAGGGAAGAGCGCGGTGGGGGCGCC
CCGGCCCCGCTGCCCTGGGGCTATGGCCATGGTGACCGGCGGCTGGGGCG
GCCCCGGCGGCGACACGAACGGCGTGGACAAGGCGGGCGGCTACCCGCGC
GCGGCCGAGGACGACTCGGCCTCGCCCCCCGGTGCCGCCAGCGACGCCGA
GCCGGGCGACGAGGAGCGGCCGGGGCTGCAGGTGGACTGCGTGGTGTGCG
GGGACAAGTCGAGCGGCAAGCATTACGGTGTCTTCACCTGCGAGGGCTGC
AAGAGCTTTTTCAAGCGAAGCATCCGCCGCAACCTCAGCTACACCTGCCG
GTCCAACCGTGACTGCCAGATCGACCAGCACCACCGGAACCAGTGCCAGT
ACTGCCGTCTCAAGAAGTGCTTCCGGGTGGGCATGAGGAAGGAGGCGGTG
CAGCGCGGCCGCATCCCGCACTCGCTGCCTGGTGCCGTGGCCGCCTCCTC
GGGCAGCCCCCCGGGCTCGGCGCTGGCGGCAGTGGCGAGCGGCGGAGACC
TCTTCCCGGGGCAGCCGGTGTCCGAACTGATCGCGCAGCTGCTGCGCGCT
GAGCCCTACCCTGCGGCGGCCGGACGCTTCGGCGCAGGGGGCGGCGCGGC
GGGCGCGGTGCTGGGCATCGACAACGTGTGCGAGCTGGCGGCGCGGCTGC
TCTTCAGCACCGTGGAGTGGGCGCGCCACGCGCCCTTCTTCCCCGAGCTG
CCGGTGGCCGACCAGGTGGCGCTGCTGCGCCTGAGCTGGAGCGAGCTCTT
CGTGCTGAACGCGGCGCAGGCGGCGCTGCCCCTGCACACGGCGCCGCTAC
TGGCCGCCGCCGGCCTCCACGCCGCGCCTATGGCCGCCGAGCGCGCCGTG
GCTTTCATGGACCAGGTGCGCGCCTTCCAGGAGCAGGTGGACAAGCTGGG
CCGCCTGCAGGTCGACTCGGCCGAGTATGGCTGCCTCAAGGCCATCGCGC
TCTTCACGCCCGACGCCTGTGGCCTCTCAGACCCGGCCCACGTTGAGAGC
CTGCAGGAGAAGGCGCAGGTGGCCCTCACCGAGTATGTGCGGGCGCAGTA
CCCGTCCCAGCCCCAGCGCTTCGGGCGCCTGCTGCTGCGGCTCCCCGCCC
TGCGCGCGGTCCCTGCCTCCCTCATCTCCCAGCTGTTCTTCATGCGCCTG
GTGGGGAAGACGCCCATTGAGACACTGATCAGAGACATGCTGCTGTCGGG
GAGTACCTTCAACTGGCCCTACGGCTCGGGCCAGTGACCATGACGGGGCC
ACGTGTGCTGTGGCCAGGCCTGCAGACAGACCTCAAGGGACAGGGAATGC
TGAGGCCTCGAGGGGCCTCCCGGGGCCCAGGACTCTGGCTTCTCTCCTCA
GACTTCTATTTTTTAAAGACTGTGAAATGTTTGTCTTTTCTGTTTTTTAA
ATGATCATGAAACCAAAAAGAGACTGATCATCCAGGCCTCAGCCTCATCC
TCCCCAGGACCCCTGTCCAGGATGGAGGGTCCAATCCTAGGACAGCCTTG
TTCCTCAGCACCCCTAGCATGAACTTGTGGGATGGTGGGGTTGGCTTCCC
TGGCATGATGGACAAAGGCCTGGCGTCGGCCAGAGGGGCTGCTCCAGTGG
GCAGGGGTAGCTAGCGTGTGCCAGGCAGATCCTCTGGACACGTAACCTAT
GTCAGACACTACATGATGACTCAAGGCCAATAATAAAGACATTTCCTACC
TGCA

SEQ ID NO: 2
Mus musculus nuclear receptor subfamily 2, group F, member 6 (Nr2f6), mRNA NCBI Reference Sequence: NM_{—}010150.2
>gi|112807198|ref|NM_{—}010150.2| Mus musculus nuclear receptor subfamily 2, group F, member 6 (Nr2f6), mRNA

GGCGCCGATGGAACGCGGGTGTCAGGCCGGCCGCAGCGCGGGGCCGGCGG
CGAGCGCCAGGGCGAGGCCGAGGCTCGGGCCCAGGCGCAGGCCGAGGCCG
GCCGCGCGAGCGCTCGGCGGGGAGACGATCCAGGGAAGGCCGCGGGTCGC
ACTCTCCACTCAGCTCTATCGCCTGGACCTCTGCGATTACGGCCGGGCGC
GCGCGGCGTGCGGGACTCCGGGTCTCCGACGCGCGCTCCCGCCGCCCCTC
CCCCCTCGCCGCGTAACTTGCGGCCAAAGTTTCCCCCCGGGCTCGGGGGC
GCCCGCGCGCGCTCGGATGGTGAGCCACTAAGTTGGCCTGGGCGGCGGGG
CCGGGCCATGGCCCCCGCGACGCTACCGGGTCCCCAGGACTCCGGACCAC
GGGACCTGGGCGCCCCAGACTCGCGCCTCTAGCGCGCCCCCGTCGACCGC
GGGCACGCGTGGGAAAGTTGGCCTGGAACCGGCCCGACCAGTTCCTGCCT
GGCGCGCGGACCGGCCGCAGGAAGTTGCCGCAAAACTTTTTTCAGGGGGG
TGTGCGACCGGAGCCCCCCGAGAGCGCGGGCTGCATGCGCCCGGGGTAGC
CGGGTCCCTCTCGGGTCGCCAGGCGTGCCCAGAGGGGACGGACTCGTCCC
GGGGCGTACCGGCCCCGCTGTCTCCGGGGCTATGGCCATGGTGACCGGTG
GCTGGGGCGACCCCGGAGGCGACACGAACGGCGTGGACAAGGCTGGTGGG
AGCTACCCACGCGCGACCGAGGACGATTCGGCGTCACCTCCCGGGGCGAC
CAGCGACGCGGAGCCGGGCGACGAGGAGCGTCCGGGGTTGCAGGTGGACT
GCGTGGTGTGCGGGGACAAGTCCAGTGGAAAGCATTACGGCGTGTTCACC
TGCGAGGGCTGCAAGAGTTTCTTCAAGCGCAGCATCCGCCGCAATCTCAG
CTACACCTGCCGGTCCAACCGTGACTGTCAGATTGATCAGCACCACCGGA
ACCAGTGTCAGTACTGTCGGCTCAAGAAGTGCTTCCGGGTGGGCATGCGC
AAGGAGGCCGTGCAGCGAGGCCGCATCCCGCATGCGCTCCCCGGTCCAGC
GGCCTGCAGTCCCCCGGGCGCGACGGGCGTCGAACCTTTCACGGGGCCGC
CAGTGTCCGAGCTGATTGCGCAGCTGCTGCGTGCTGAGCCCTACCCCGCG
GCCGGACGCTTTGGTGGCGGCGGCGCTGTACTGGGCATCGACAACGTGTG
CGAGTTGGCGGCACGCCTGCTGTTCAGCACGGTCGAGTGGGCCCGCCACG
CGCCCTTCTTCCCCGAGCTGCCGGCCGCCGACCAGGTGGCGCTGCTGCGG
CTCAGCTGGAGTGAGCTCTTCGTGCTGAACGCGGCGCAGGCGGCGCTGCC
GCTGCATACGGCACCGCTGCTGGCCGCCGCGGGGTTGCATGCCGCGCCCA
TGGCAGCCGAGCGGGCCGTGGCCTTCATGGACCAGGTGCGTGCCTTCCAG
GAGCAGGTGGACAAGCTGGGCCGCCTGCAGGTGGATGCTGCGGAGTACGG
CTGCCTCAAGGCCATCGCGCTCTTCACGCCTGATGCCTGTGGCCTTTCTG
ACCCAGCCCATGTGGAGAGCCTGCAGGAGAAGGCACAGGTGGCCCTCACC
GAGTATGTGCGTGCCCAGTACCCATCGCAGCCCCAGCGCTTTGGGCGTCT
GCTGCTGCGGCTGCCAGCCCTGCGTGCTGTGCCCGCATCCCTCATCTCCC
AGCTCTTCTTCATGCGCCTGGTGGGCAAGACACCCATCGAGACCCTCATC
CGGGACATGCTTCTGTCAGGGAGCACCTTTAACTGGCCCTATGGCTCGGG
CTAGTGATAGTCACCTTCCAGGACATACATGGAAACTGGGGCCTTGTGGG
GACCCTGGGGATCAGGGCCCCAGCTTCTCTTTTGAGACTGATTTCTTTTT
TTAAAGACTGTGAAATGTTTGTTTTGTTTTATTTTTTAAATAATCATGAA
ACCAAAAAGATTTGGATCTCCCAGGCCTTGTCCTGGCAGACCTTCAACAG
TCTGGAGCCAGCATGCTGATGCCTCTGGTGTCATGGGTATCTGGAAAGGC
CACTGCAGCTAGGCAGGAGTACTATGGGCCAGGAGGATCCCCTGGATACA
TGGTCCACGGAGGGCACCATGGGATGATGAAAACCTGGCCAATAATAAAG
GTATTCCCTTACTTGGTC

SEQ ID NO: 3
Protein Sequence of Human NR2F6
>gi|23503053|sp|P10588.2|NR2F6_HUMAN RecName: Full=Nuclear receptor subfamily 2 group F member 6; AltName: Full=V-erbA-related protein 2; Short=EAR-2

MAMVTGGWGGPGGDTNGVDKAGGYPRAAEDDSASPPGAASDAEPGDEERP
GLQVDCVVCGDKSSGKHYGVFTCEGCKSFFKRSIRRNLSYTCRSNRDCQI
DQHHRNQCQYCRLKKCFRVGMRKEAVQRGRIPHSLPGAVAASSGSPPGSA
LAAVASGGDLFPGQPVSELIAQLLRAEPYPAAAGRFGAGGGAAGAVLGID
NVCELAARLLFSTVEWARHAPFFPELPVADQVALLRLSWSELFVLNAAQA
ALPLHTAPLLAAAGLHAAPMAAERAVAFMDQVRAFQEQVDKLGRLQVDSA
EYGCLKAIALFTPDACGLSDPAHVESLQEKAQVALTEYVRAQYPSQPQRF
GRLLLRLPALRAVPASLISQLFFMRLVGKTPIETLIRDMLLSGSTFNWPY
GSGQ

SEQ ID NO: 4
Protein Sequence of NR2F6 Mus Musculus
>gi|112807199|ref|NP_{—}034280.2| nuclear receptor subfamily 2 group F member 6 [Mus musculus]

MAMVTGGWGDPGGDTNGVDKAGGSYPRATEDDSASPPGATSDAEPGDEER
PGLQVDCVVCGDKSSGKHYGVFTCEGCKSFFKRSIRRNLSYTCRSNRDCQ
IDQHHRNQCQYCRLKKCFRVGMRKEAVQRGRIPHALPGPAACSPPGATGV
EPFTGPPVSELIAQLLRAEPYPAAGRFGGGGAVLGIDNVCELAARLLFST
VEWARHAPFFPELPAADQVALLRLSWSELFVLNAAQAALPLHTAPLLAAA
GLHAAPMAAERAVAFMDQVRAFQEQVDKLGRLQVDAAEYGCLKAIALFTP
DACGLSDPAHVESLQEKAQVALTEYVRAQYPSQPQRFGRLLLRLPALRAV
PASLISQLFFMRLVGKTPIETLIRDMLLSGSTFNWPYGSG
SEQ ID NO: 18
(human siRNA) GCCGUCUCAAGAAGUGCUU
SEQ ID NO: 19
(human siRNA) CAUUGAGACACUGAUCAGA
SEQ ID NO: 20
(human siRNA) GCAAGCAUUACGGUGUCUU
SEQ ID NO: 21
(human siRNA) CCCCUAGCAUGAACUUGUG
(mus shNR2F6.1)
SEQ ID NO: 5
GAT CCG CAT TAC GGC GTG TTC ACC TTC AAG AGA
GGT GAA CAC GCC GTA ATG CTT TTT TCT AGA G
(mus shNR2F6.2)
SEQ ID NO: 6
GAT CCG CAA CCG TGA CTG TCA GAT TAA GTT CTC
TAA TCT GAC AGT CAC GGT TGT TTT TTC TAG AG
(mus shNR2F6.3)
SEQ ID NO: 7
GAT CCG TGT CCG AGC TGA TTG CGC ATT CAA GAG
ATG CGC AAT CAG CTC GGA CAT TTT TTC TAG AG
(human shNR2F6.1)
SEQ ID NO: 8
GAT CCG CAT TAC GGT GTC TTC ACC TTC AAG AGA
GGT GAA GAC ACC GTA ATG CTT TTT TCT AGA G
(human shNR2F6.2)
SEQ ID NO: 9
GAT CCG CCT CTG GAC ACG TAA CCT ATT CAA GAG
ATA GGT TAC GTG TCC AGA GGT TTT TTC TAG AG

### Primers

Human NR2F6:
SEQ ID NO: 10
Fwd: 5′-TCTCCCAGCTGTTCTTCATGC-3′
SEQ ID NO: 11
Revs: 5′-CCAGTTGAAGGTACTCCCCG-3′
Human GAPDH:
SEQ ID NO: 12
Fwd: 5′-GGCCTCCAAGGAGTAAGACC-3′
SEQ ID NO: 13
Revs: 5′-AGGGGTCTACATGGCAACTG-3′.
3′ end Mus NR2F6:
SEQ ID NO: 14
Fwd: 5′-CCTGGCAGACCTTCA ACAG-3′
SEQ ID NO: 15
Revs: 5′-GATCCTCCTGGCCCATAGT-3′
3′ end Mus L32:
SEQ ID NO: 16
Fwd: 5′-GCCATCAGAGTCACCAATCC-3′
SEQ ID NO: 17
Revs: 5′-AAACATGCACACAAGCCATC-3′

### REFERENCES

1. Ichim, C. V. and R. A. Wells, First among equals: The cancer cell hierarchy. Leukemia and Lymphoma (in press), 2006.
2. Bonnet, D. and J. E. Dick, Human acute myeloid leukemia is organized as a hierarchy that originates from a primitive hematopoietic cell. Nat Med, 1997. 3(7): p. 730-7.
3. Singh, S. K., et al., Identification of a cancer stem cell in human brain tumors. Cancer Res, 2003. 63(18): p. 5821-8.
4. Singh, S. K., et al., Identification of human brain tumour initiating cells. Nature, 2004. 432(7015): p. 396-401.
5. Al-Hajj, M., et al., Prospective identification of tumorigenic breast cancer cells. Proc Natl Acad Sci U S A, 2003. 100(7): p. 3983-8.
6. Reya, T., et al., A role for Wnt signalling in self-renewal of haematopoietic stem cells. Nature, 2003. 423(6938): p. 409-14.
7. Stier, S., et al., Notchl activation increases hematopoietic stem cell self-renewal in vivo and favors lymphoid over myeloid lineage outcome. Blood, 2002. 99(7): p. 2369-78.
8. Karlsson, G., et al., Smad4 is critical for self-renewal of hematopoietic stem cells. J Exp Med, 2007. 204(3): p. 467-74.
9. Schnabel, C. A., Y. Jacobs, and M. L. Cleary, HoxA9-mediated immortalization of myeloid progenitors requires functional interactions with TALE cofactors Pbx and Meis. Oncogene, 2000. 19(5): p. 608-16.
10. Thorsteinsdottir, U., et al., Overexpression of the myeloid leukemia-associated Hoxa9 gene in bone marrow cells induces stem cell expansion. Blood, 2002. 99(1): p. 121-9.
11. Thorsteinsdottir, U., et al., Overexpression of HOXA10 in murine hematopoietic cells perturbs both myeloid and lymphoid differentiation and leads to acute myeloid leukemia. Mol Cell Biol, 1997. 17(1): p. 495-505.
12. Sauvageau, G., et al., Overexpression of HOXB4 in hematopoietic cells causes the selective expansion of more primitive populations in vitro and in vivo. Genes Dev, 1995. 9(14): p. 1753-65.
13. Lessard, J. and G. Sauvageau, Bmi-1 determines the proliferative capacity of normal and leukaemic stem cells. Nature, 2003. 423(6937): p. 255-60.
14. Park, I. K., et al., Bmi-1 is required for maintenance of adult self-renewing haematopoietic stem cells. Nature, 2003. 423(6937): p. 302-5.
15. Ernst, P., et al., Definitive hematopoiesis requires the mixed-lineage leukemia gene. Dev Cell, 2004. 6(3): p. 437-43.
16. Heyworth, C., et al., A GATA-2/estrogen receptor chimera functions as a ligand-dependent negative regulator of self-renewal. Genes Dev, 1999. 13(14): p. 1847-60.
17. Zeng, H., et al., Transcription factor Gfi1 regulates self-renewal and engraftment of hematopoietic stem cells. Embo J, 2004. 23(20): p. 4116-25.
18. Steidl, U., et al., Essential role of Jun family transcription factors in PU.1 knockdown-induced leukemic stem cells. Nat Genet, 2006. 38(11): p. 1269-77.
19. Iwasaki, H., et al., Distinctive and indispensable roles of PU.1 in maintenance of hematopoietic stem cells and their differentiation. Blood, 2005. 106(5): p. 1590-600.
20. White, J. R. and K. Weston, Myb is required for self-renewal in a model system of early hematopoiesis. Oncogene, 2000. 19(9): p. 1196-205.
21. Rebel, V. I., et al., Distinct roles for CREB-binding protein and p300 in hematopoietic stem cell self-renewal. Proc Natl Acad Sci U S A, 2002. 99(23): p. 14789-94.
22. Wilson, A., et al., c-Myc controls the balance between hematopoietic stem cell self-renewal and differentiation. Genes Dev, 2004. 18(22): p. 2747-63.
23. Galan-Caridad, J.M., et al., Zfx controls the self-renewal of embryonic and hematopoietic stem cells. Cell, 2007. 129(2): p. 345-57.
24. Zon, L. I., Intrinsic and extrinsic control of haematopoietic stem-cell self-renewal. Nature, 2008. 453(7193): p. 306-13.
25. Nerlov, C. and T. Graf, PU.1 induces myeloid lineage commitment in multipotent hematopoietic progenitors. Genes Dev, 1998. 12(15): p. 2403-12.
26. Nerlov, C., et al., GATA-1 interacts with the myeloid PU.1 transcription factor and represses PU.1-dependent transcription. Blood, 2000. 95(8): p. 2543-51.
27. Lapidot, T., et al., A cell initiating human acute myeloid leukaemia after transplantation into SCID mice. Nature, 1994. 367(6464): p. 645-8.
28. Blair, A., D. E. Hogge, and H. J. Sutherland, Most acute myeloid leukemia progenitor cells with long-term proliferative ability in vitro and in vivo have the phenotype CD34(+)/CD71(−)/HLA-DR. Blood, 1998. 92(11): p. 4325-35.
29. de Lima, M., et al., Implications of potential cure in acute myelogenous leukemia: development of subsequent cancer and return to work. Blood, 1997. 90(12): p. 4719-24.

All references listed herein are expressly incorporated by reference in their entireties. The invention may be embodied in other specific forms besides and beyond those described herein. The foregoing embodiments are therefore to be considered in all respects illustrative rather than limiting, and the scope of the invention is defined and limited only by the appended claims and their equivalents, rather than by the foregoing description.

1. A method of treating cancer comprising: indentifying a subject suffering from a cancerous condition; administering to the subject an effective amount of a composition comprising a synthetic oligonucleotide complementary to a nuclear receptor having a mRNA sequence of at least 75% sequence identity to the mRNA sequence of SEQ ID NO: 1 that induces the RNA interference, wherein said nucleotide comprises a sense oligonucleotide strand and an antisense oligonucleotide strand, wherein the sense and antisense oligonucleotide strands form a duplex, and wherein the sense oligonucleotide strand comprises a portion of SEQ ID NO:1 that has been selected based on its ability to inhibits the expression of the nuclear receptor NR2F6 by causing degradation of a ribonucleic acid encoding nuclear receptor NR2F6 by activation of RNA interference.

2. The method of claim 1 wherein the synthetic oligonucleotide consists of a short-interfering ribonucleic acid (siRNA) molecule.

3. The method of claim 1 wherein the synthetic oligonucleotide consists of a short-hairpin ribonucleic acid (shRNA) molecule.

4. The method of claim 1 wherein the synthetic oligonucleotide consists of an antisense ribonucleic acid molecule.

5. The method of inhibiting expression of NR2F6 protein in a subject for a therapeutic purpose, comprising: identifying a subject in need of NR2F6 inhibition; administering to said subject an effective amount of pharmaceutical composition comprising a synthetic oligonucleotide comprising a sense strand and an antisense strand, wherein the sense and antisense strands form a duplex, and wherein the sense RNA strand comprises SEQ ID NO:1, thereby specifically inhibiting the expression of NR2F6.

6. The method of claim 5, wherein the pharmaceutical composition further comprises a delivery agent.

7. The method of claim 6, wherein the delivery agent comprises a liposome.

8. A method of inhibiting tumor growth, comprising: identifying a subject suffering from a tumor growth, contacting the tumor with an oligonucleic acid comprising a sense oligonucleotide strand and an antisense oligonucloetide strand, wherein the sense and antisense oligonucleotide strands form a synthetic oligonucleotide duplex, and wherein the sense oligonucloetide strand comprises a portion of SEQ ID NO:1 selected for its ability to silence the expression of NR2F6, thereby specifically inhibiting the expression of NR2F6 in the tumor and thus inhibiting growth of the tumor.

9. The method of claim 8, wherein the step of contacting the tumor with the siRNA results in at least one of an induction of differentiation or decreased cancer stem cell activity indicated by a decrease in one of the following self-renewal, growth, proliferation, differentiation and programmed cell death in mammalian cells.

10. The method of claim 9 wherein the effective portion of the oligonucleotide consists of SEQ ID NO: 18.

11. The method of claim 9 wherein the effective portion of the oligonucleotide consists of SEQ ID NO: 19.

12. The method of claim 9 wherein the effective portion of the oligonucleotide consists of SEQ ID NO: 20.

13. A composition comprising an oligonucleotide complementary to a nuclear receptor having a mRNA sequence of at least 75% sequence identity to the mRNA sequence of SEQ ID NO: 1, wherein said oligonucleotide comprises a sense oligonucleotide strand and an antisense oligonucleotide strand, wherein the sense and antisense oligonucleotide strands form a duplex, and wherein the sense oligonucleotide strand comprises a portion of SEQ ID NO:1 that is selected based on its ability to inhibits the expression of the nuclear receptor NR2F6 by causing degradation of a ribonucleic acid encoding nuclear receptor NR2F6.

14. The composition of claim 13 wherein the oligonucleotide is a short-interfering ribonucleic acid (siRNA) molecule.

15. The composition of claim 13 wherein the oligonucleotide is a short-hairpin ribonucleic acid (shRNA) molecule.

16. The composition of claim 13 wherein the oligonucleotide is an antisense ribonucleic acid molecule.

17. The pharmaceutical composition of claim 13, wherein the oligonucleotide is selected from the group consisting of: a short-interfering ribonucleic acid (siRNA) molecule, short-hairpin ribonucleic acid (shRNA) molecule, and an antisense ribonucleic acid molecule.

18. The pharmaceutical composition of claim 13 further comprising at least one additional chemotherapeutic agent.

19. The pharmaceutical composition of claims 13 further comprising a delivery agent.

20. The pharmaceutical composition of claim 19, wherein the delivery agent comprises a liposome.

21. The method of claim 9 wherein the effective portion of the oligonucleotide consists of SEQ ID NO: 21.


SEQUENCE LISTING (21 sequences)

SEQ ID NO: 1 - DNA, 1804 bp, Homo Sapiens
gtgcagcccgtgccccccgcgcgccggggccgaatgcgcgccgcgtagggtcccccgggccgagaggggtgcccggagggaagagcgcggtgggggcgccccggccccgctgccctggggctatggccatggtgaccggcggctggggcggccccggcggcgacacgaacggcgtggacaaggcgggcggctacccgcgcgcggccgaggacgactcggcctcgccccccggtgccgccagcgacgccgagccgggcgacgaggagcggccggggctgcaggtggactgcgtggtgtgcggggacaagtcgagcggcaagcattacggtgtcttcacctgcgagggctgcaagagctttttcaagcgaagcatccgccgcaacctcagctacacctgccggtccaaccgtgactgccagatcgaccagcaccaccggaaccagtgccagtactgccgtctcaagaagtgcttccgggtgggcatgaggaaggaggcggtgcagcgcggccgcatcccgcactcgctgcctggtgccgtggccgcctcctcgggcagccccccgggctcggcgctggcggcagtggcgagcggcggagacctcttcccggggcagccggtgtccgaactgatcgcgcagctgctgcgcgctgagccctaccctgcggcggccggacgcttcggcgcagggggcggcgcggcgggcgcggtgctgggcatcgacaacgtgtgcgagctggcggcgcggctgctcttcagcaccgtggagtgggcgcgccacgcgcccttcttccccgagctgccggtggccgaccaggtggcgctgctgcgcctgagctggagcgagctcttcgtgctgaacgcggcgcaggcggcgctgcccctgcacacggcgccgctactggccgccgccggcctccacgccgcgcctatggccgccgagcgcgccgtggctttcatggaccaggtgcgcgccttccaggagcaggtggacaagctgggccgcctgcaggtcgactcggccgagtatggctgcctcaaggccatcgcgctcttcacgcccgacgcctgtggcctctcagacccggcccacgttgagagcctgcaggagaaggcgcaggtggccctcaccgagtatgtgcgggcgcagtacccgtcccagccccagcgcttcgggcgcctgctgctgcggctccccgccctgcgcgcggtccctgcctccctcatctcccagctgttcttcatgcgcctggtggggaagacgcccattgagacactgatcagagacatgctgctgtcggggagtaccttcaactggccctacggctcgggccagtgaccatgacggggccacgtgtgctgtggccaggcctgcagacagacctcaagggacagggaatgctgaggcctcgaggggcctcccggggcccaggactctggcttctctcctcagacttctattttttaaagactgtgaaatgtttgtcttttctgttttttaaatgatcatgaaaccaaaaagagactgatcatccaggcctcagcctcatcctccccaggacccctgtccaggatggagggtccaatcctaggacagccttgttcctcagcacccctagcatgaacttgtgggatggtggggttggcttccctggcatgatggacaaaggcctggcgtcggccagaggggctgctccagtgggcaggggtagctagcgtgtgccaggcagatcctctggacacgtaacctatgtcagacactacatgatgactcaaggccaataataaagacatttcctacctgca

SEQ ID NO: 2 - DNA, 2218 bp, Mus musculus
ggcgccgatggaacgcgggtgtcaggccggccgcagcgcggggccggcggcgagcgccagggcgaggccgaggctcgggcccaggcgcaggccgaggccggccgcgcgagcgctcggcggggagacgatccagggaaggccgcgggtcgcactctccactcagctctatcgcctggacctctgcgattacggccgggcgcgcgcggcgtgcgggactccgggtctccgacgcgcgctcccgccgcccctcccccctcgccgcgtaacttgcggccaaagtttccccccgggctcgggggcgcccgcgcgcgctcggatggtgagccactaagttggcctgggcggcggggccgggccatggcccccgcgacgctaccgggtccccaggactccggaccacgggacctgggcgccccagactcgcgcctctagcgcgcccccgtcgaccgcgggcacgcgtgggaaagttggcctggaaccggcccgaccagttcctgcctggcgcgcggaccggccgcaggaagttgccgcaaaacttttttcaggggggtgtgcgaccggagccccccgagagcgcgggctgcatgcgcccggggtagccgggtccctctcgggtcgccaggcgtgcccagaggggacggactcgtcccggggcgtaccggccccgctgtctccggggctatggccatggtgaccggtggctggggcgaccccggaggcgacacgaacggcgtggacaaggctggtgggagctacccacgcgcgaccgaggacgattcggcgtcacctcccggggcgaccagcgacgcggagccgggcgacgaggagcgtccggggttgcaggtggactgcgtggtgtgcggggacaagtccagtggaaagcattacggcgtgttcacctgcgagggctgcaagagtttcttcaagcgcagcatccgccgcaatctcagctacacctgccggtccaaccgtgactgtcagattgatcagcaccaccggaaccagtgtcagtactgtcggctcaagaagtgcttccgggtgggcatgcgcaaggaggccgtgcagcgaggccgcatcccgcatgcgctccccggtccagcggcctgcagtcccccgggcgcgacgggcgtcgaacctttcacggggccgccagtgtccgagctgattgcgcagctgctgcgtgctgagccctaccccgcggccggacgctttggtggcggcggcgctgtactgggcatcgacaacgtgtgcgagttggcggcacgcctgctgttcagcacggtcgagtgggcccgccacgcgcccttcttccccgagctgccggccgccgaccaggtggcgctgctgcggctcagctggagtgagctcttcgtgctgaacgcggcgcaggcggcgctgccgctgcatacggcaccgctgctggccgccgcggggttgcatgccgcgcccatggcagccgagcgggccgtggccttcatggaccaggtgcgtgccttccaggagcaggtggacaagctgggccgcctgcaggtggatgctgcggagtacggctgcctcaaggccatcgcgctcttcacgcctgatgcctgtggcctttctgacccagcccatgtggagagcctgcaggagaaggcacaggtggccctcaccgagtatgtgcgtgcccagtacccatcgcagccccagcgctttgggcgtctgctgctgcggctgccagccctgcgtgctgtgcccgcatccctcatctcccagctcttcttcatgcgcctggtgggcaagacacccatcgagaccctcatccgggacatgcttctgtcagggagcacctttaactggccctatggctcgggctagtgatagtcaccttccaggacatacatggaaactggggccttgtggggaccctggggatcagggccccagcttctcttttgagactgatttctttttttaaagactgtgaaatgtttgttttgttttattttttaaataatcatgaaaccaaaaagatttggatctcccaggccttgtcctggcagaccttcaacagtctggagccagcatgctgatgcctctggtgtcatgggtatctggaaaggccactgcagctaggcaggagtactatgggccaggaggatcccctggatacatggtccacggagggcaccatgggatgatgaaaacctggccaataataaaggtattcccttacttggtc

SEQ ID NO: 3 - protein, 404 aa, Homo sapien
MAMVTGGWGGPGGDTNGVDKAGGYPRAAEDDSASPPGAASDAEPGDEERPGLQVDCVVCGDKSSGKHYGVFTCEGCKSFFKRSIRRNLSYTCRSNRDCQIDQHHRNQCQYCRLKKCFRVGMRKEAVQRGRIPHSLPGAVAASSGSPPGSALAAVASGGDLFPGQPVSELIAQLLRAEPYPAAAGRFGAGGGAAGAVLGIDNVCELAARLLFSTVEWARHAPFFPELPVADQVALLRLSWSELFVLNAAQAALPLHTAPLLAAAGLHAAPMAAERAVAFMDQVRAFQEQVDKLGRLQVDSAEYGCLKAIALFTPDACGLSDPAHVESLQEKAQVALTEYVRAQYPSQPQRFGRLLLRLPALRAVPASLISQLFFMRLVGKTPIETLIRDMLLSGSTFNWPYGSGQ

SEQ ID NO: 4 - protein, 390 aa, Mus musculus
MAMVTGGWGDPGGDTNGVDKAGGSYPRATEDDSASPPGATSDAEPGDEERPGLQVDCVVCGDKSSGKHYGVFTCEGCKSFFKRSIRRNLSYTCRSNRDCQIDQHHRNQCQYCRLKKCFRVGMRKEAVQRGRIPHALPGPAACSPPGATGVEPFTGPPVSELIAQLLRAEPYPAAGRFGGGGAVLGIDNVCELAARLLFSTVEWARHAPFFPELPAADQVALLRLSWSELFVLNAAQAALPLHTAPLLAAAGLHAAPMAAERAVAFMDQVRAFQEQVDKLGRLQVDAAEYGCLKAIALFTPDACGLSDPAHVESLQEKAQVALTEYVRAQYPSQPQRFGRLLLRLPALRAVPASLISQLFFMRLVGKTPIETLIRDMLLSGSTFNWPYGSG

SEQ ID NO: 5 - DNA, 64 bp, artificial
  mus shNR2F6.1
gatccgcattacggcgtgttcaccttcaagagaggtgaacacgccgtaatgcttttttctagag

SEQ ID NO: 6 - DNA, 65 bp, artificial
  mus shNR2F6.2
gatccgcaaccgtgactgtcagattaagttctctaatctgacagtcacggttgttttttctagag

SEQ ID NO: 7 - DNA, 65 bp, artificial
  mus shNR2F6.3
gatccgtgtccgagctgattgcgcattcaagagatgcgcaatcagctcggacattttttctagag

SEQ ID NO: 8 - DNA, 64 bp, artificial
  human shNR2F6.1
gatccgcattacggtgtcttcaccttcaagagaggtgaagacaccgtaatgcttttttctagag

SEQ ID NO: 9 - DNA, 65 bp, artificial
  human shNR2F6.2
gatccgcctctggacacgtaacctattcaagagataggttacgtgtccagaggttttttctagag

SEQ ID NO: 10 - DNA, 21 bp, artificial
  Primer Human NR2F6 Fwd
tctcccagctgttcttcatgc

SEQ ID NO: 11 - DNA, 20 bp, artificial
  Primer Human NR2F6 Rev
ccagttgaaggtactccccg

SEQ ID NO: 12 - DNA, 20 bp, artificial
  Primer Human GAPDH Fwd
ggcctccaaggagtaagacc

SEQ ID NO: 13 - DNA, 20 bp, artificial
  Primer Human GAPDH Rev
aggggtctacatggcaactg

SEQ ID NO: 14 - DNA, 19 bp, artificial
  Primer 3′ end Mus NR2F6 Fwd
cctggcagaccttcaacag

SEQ ID NO: 15 - DNA, 19 bp, artificial
  Primer 3′ end Mus NR2F6 Revs
gatcctcctggcccatagt

SEQ ID NO: 16 - DNA, 20 bp, artificial
  Primer 3′ end Mus L32 Fwd
gccatcagagtcaccaatcc

SEQ ID NO: 17 - DNA, 20 bp, artificial
  Primer 3′ end Mus L32 Rev
aaacatgcacacaagccatc

SEQ ID NO: 18 - RNA, 19 nt, artificial
  human siRNA
gccgucucaagaagugcuu

SEQ ID NO: 19 - RNA, 19 nt, artificial
  human siRNA
cauugagacacugaucaga

SEQ ID NO: 20 - RNA, 19 nt, artificial
  human siRNA
gcaagcauuacggugucuu

SEQ ID NO: 21 - RNA, 19 nt, artificial
  human siRNA
ccccuagcaugaacuugug